(19) United States
(12) Patent Application Publication
(10) Pub. No.: US 2009/0318774 A1
(43) Pub. Date: Dec. 24, 2009

(54) VEHICLE, AND PHYSIOLOGICAL INFORMATION GATHERING SYSTEM WITH THE SAME

(75) Inventors: Shinji Ichikawa, Toyota-shi (JP); Tetsuhiro Ishikawa, Toyota-shi (JP)

Correspondence Address: OLIFF & BERRIDGE, PLC, P.O. BOX 320850, ALEXANDRIA, VA, 22320-4850, US

(73) Assignee: TOYOTA JIDOSHA KABUSHIKI KAISHA, TOYOTA-SHI (JP)

(21) Appl. No.: 12/310,045

(22) PCT Filed: Jul. 26, 2007
(86) PCT No.: PCT/JP2007/065121

§ 371 Date: Feb. 9, 2009

(30) Foreign Application Priority Data
Sep. 11, 2006 (JP) 2006-245394

(51) Int. Cl. A61B 5/00

(52) U.S. Cl. 600/300

(57) ABSTRACT

Sensors detect physiological data of an occupant of a vehicle. A storage device accumulates the physiological data of the occupant detected by each of the sensors for storage. When a charging cable for charging a power storage device in a motive power output device is connected, a vehicle ECU transmits the physiological data stored in the storage device to an element external to the vehicle through electric power lines and the charging cable, employing a modem.

### TECHNICAL FIELD

The present invention relates to a physiological information gathering system employing a vehicle having mounted thereon a power storage device which is rechargeable from a source external to the vehicle.

### BACKGROUND ART

A system is publicly known which is capable of evaluating the condition of a driver according to physiological parameters of the driver measured during travel in a vehicle.

For example, Japanese National Patent Publication No. 2004-507308 discloses a method and a device for diagnosing the driver's fitness to drive. This method and device for diagnosing are based on the combination of driver's physiological measured values obtained during travel in the vehicle and the driver's health-relevant data measured stationarily at the driver's home area. By means of an expert system, deviations of the driver's condition are weighted with parameters indicating the stress on the driver and are interpreted.

According to this method and device for diagnosing, a warning can be output to the driver based on the result of diagnosis made by the expert system. In case of emergency, auxiliary measures can be initiated.

Since an occupant is restrained in the same position on his/her seat for a long time during travel of a vehicle, a large amount of physiological data (e.g., blood pressure, heart rate) of the occupant can be gathered with stability. If such a large amount of gathered physiological data can be utilized at a medical institution or the like external to the vehicle, time to gather such physiological data at medical institutions can be saved. Further, a precise diagnosis can also be made based on the large amount of physiological data.

The aforementioned Japanese National Patent Publication No. 2004-507308 discloses detecting the deviations of the driver's condition during travel and outputting a warning to the driver in case of emergency, but fails to disclose specific measures to gather a large amount of physiological data while the occupant is aboard the vehicle, restrained on his/her seat for a long time, and use the data at a place (e.g., a medical institution) external to the vehicle.

### DISCLOSURE OF THE INVENTION

It is therefore an object of the present invention to provide a vehicle that gathers physiological data of an occupant and allows the gathered physiological data to be used at an element external to the vehicle, as well as a physiological information gathering system employing the same.

According to the present invention, a vehicle includes a power storage device which is rechargeable, an electric power input part, a voltage conversion device, a detection device, a storage device, a communication device and a control unit. The electric power input part is provided to input electric power for charging the power storage device from a source external to the vehicle. The voltage conversion device is configured to allow the electric power received through the electric power input part to be converted into a voltage level of the power storage device for output to the power storage device. The detection device detects physiological data of an occupant of the vehicle. The storage device accumulates the physiological data detected by the detection device for storage. The communication device is configured to allow communication with an element external to the vehicle through the electric power input part. The control unit transmits the physiological data accumulated in the storage device to the element external to the vehicle through the electric power input part, employing the communication device.

The power storage device is a device that can accumulate electric power, and includes not only a secondary battery but also a capacitor. An occupant includes not only the driver but also a passenger other than the driver. Physiological data detected by the detection device includes, for example, blood pressure, heart rate, body fat percentage and body temperature of the occupant. The detection device detecting physiological data includes not only a contact-type one installed in the steering wheel, the shift lever, a door knob or the like, but also a noncontact-type one employing infrared, images or the like.

Preferably, the detection device continually detects the physiological data while the occupant is seated on a seat. When the power storage device is charged by the electric power input through the electric power input part, the control unit collectively transmits the physiological data accumulated in the storage device to the element external to the vehicle through the electric power input part, employing the communication device.

Further, according to the present invention, a physiological information gathering system includes the above-described vehicle, a power feeding device and a server. The power feeding device is configured to allow supply of electric power to the vehicle from a source external to the vehicle. The server receives physiological data transmitted out from the power input part of the vehicle through the power feeding device during charging of the power storage device of the vehicle from the power feeding device.

Preferably, the power feeding device includes an electric power line electrically connected to the electric power input part during charging of the power storage device.

In the present invention, physiological data of an occupant is detected by the detection device while the occupant is aboard the vehicle, restrained on his/her seat for a long time, and the detected physiological data is stored in the storage device. Herein, the vehicle includes an electric power input part and a voltage conversion device, and allows the power storage device to be charged from a source external to the vehicle. A large amount of physiological data accumulated in the storage device while the occupant is aboard the vehicle is transmitted to an element external to the vehicle through the electric power input part, employing the electric power input part as a communication interface with the element external to the vehicle during charging of the power storage device.

The present invention therefore enables gathering and accumulation of a large amount of physiological data of an occupant while he/she is aboard the vehicle, as well as transmission of the accumulated large amount of physiological data to an element external to the vehicle. As a consequence, the large amount of physiological data gathered in the vehicle can be used for diagnosis at a medical institution or the like.

### BRIEF DESCRIPTION OF THE DRAWINGS

FIG. 1 is an overall view of a physiological information gathering system according to an embodiment of the present invention.

FIG. 2 is a schematic configuration diagram of the vehicle shown in FIG. 1.

FIG. 3 is a functional block diagram of the motive power output device shown in FIG. 2.

FIG. 4 illustrates a zero-phase equivalent circuit of the inverters and motor generators shown in FIG. 3.

FIG. 5 is a flow chart to describe a control structure in the vehicle ECU shown in FIG. 2 during charging of a power storage device.

### BEST MODES FOR CARRYING OUT THE INVENTION

In the following, an embodiment of the present invention will be described in detail with reference to the drawings. Like reference characters denote like or corresponding parts throughout the drawings, and description thereof will not be repeated.

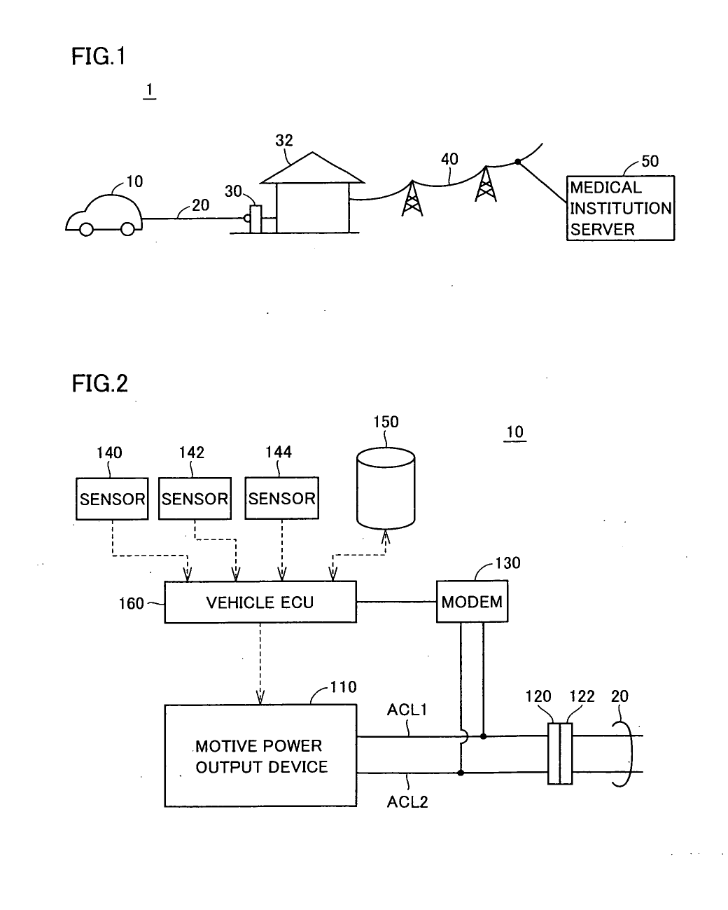

FIG. 1 is an overall view of a physiological information gathering system according to an embodiment of the present invention. Referring to FIG. 1, this physiological information gathering system 1 includes a vehicle 10, a charging cable 20, a charging station 30, a house 32, a power transmission line 40 and a medical institution server 50.

Vehicle 10 is an electrically-powered vehicle having mounted thereon a rechargeable power storage device as a DC power source. Although vehicle 10 will be described below as a hybrid vehicle, vehicle 10 may be an electric vehicle or a fuel cell vehicle.

Vehicle 10 is electrically connectable to charging station 30 through charging cable 20. Vehicle 10 can then receive a supply of system power from charging station 30 through charging cable 20 by a method which will be described later, thereby charging the on-vehicle power storage device.

Also mounted on vehicle 10 are various types of sensors that can detect various types of physiological data (e.g., blood pressure, heart rate) of an occupant (including the driver) and a storage device for accumulating their detected values for storage, so that physiological data of the occupant is gathered by the sensors while he/she is aboard the vehicle to be accumulated in the storage device. When charging cable 20 is connected to charging station 30 for charging the power storage device after arriving home, vehicle 10 outputs the physiological data accumulated in the storage device to an element external to the vehicle through charging cable 20.

Charging cable 20 is an electric power line for charging the power storage device mounted on vehicle 10 from charging station 30. Charging cable 20 also serves as a communication medium to output the physiological data of the occupant gathered and accumulated in vehicle 10 from vehicle 10 to an element external thereto.

Charging station 30 receives, from house 32, the system power supplied through power transmission line 40 to supply charging power to vehicle 10 connected through charging cable 20. House 32 supplies charging station 30 with part of the system power received through power transmission line 40.

Medical institution server 50, which is a patient management server installed in a medical institution such as a hospital, is connected to power transmission line 40 supplying the system power to house 32. Medical institution server 50 receives physiological data of the occupant of vehicle 10 being transmitted from vehicle 10 through charging cable 20, charging station 30, house 32 and power transmission line 40 in this order when vehicle 10 is connected to charging station 30 through charging cable 20. Further, medical institution server 50 transmits the result of diagnosis made based on the received physiological data and the method of treatment to house 32 through power transmission line 40.

FIG. 2 is a schematic configuration diagram of vehicle 10 shown in FIG. 1. Referring to FIG. 2, vehicle 10 includes a motive power output device 110, electric power lines ACL1 and ACL2, a connector 120, a modem 130, sensors 140, 142 and 144, a storage device 150 and a vehicle ECU (Electronic Control Unit) 160.

Motive power output device 110 outputs the driving force for this vehicle 10. According to a charge command from vehicle ECU 160, motive power output device 110 converts the charging power (system power) received through electric power lines ACL1 and ACL2 into DC power to charge its internal power storage device (not shown). The configuration of motive power output device 110 will be described later.

Electric power lines ACL1 and ACL2 provide motive power output device 110 with the charging power (system power) supplied through charging cable 20. Electric power lines ACL1 and ACL2 forward transmitted data (physiological data of the occupant gathered while he/she is aboard the vehicle) received through modem 130 to charging cable 20.

Modem 130 is connected to electric power lines ACL1 and ACL2, and according to a command from vehicle ECU 160, transmits the physiological data to medical institution server 50 (FIG. 1) through electric power lines ACL1, ACL2 and charging cable 20.

Sensors 140, 142 and 144 detect the physiological data of the occupant of vehicle 10. While three sensors are illustrated herein by way of example, the number of sensors is not limited to three, and more than three sensors may be provided. These sensors include, for example, a contact-type sensor installed in the steering wheel, the shift lever, a door knob or the like to detect, for example, blood pressure, heart rate and body fat percentage of the occupant. Alternatively, these sensors may include a noncontact-type sensor which detects body temperature of the occupant through infrared or detects the blinking state of the driver through images. Each sensor outputs its detected value to vehicle ECU 160.

Storage device 150 receives, from vehicle ECU 160, the physiological data of the occupant detected by sensors 140, 142 and 144 to accumulate and store the received physiological data. During charging of vehicle 10 from charging station 30, storage device 150 outputs the stored physiological data to vehicle ECU 160 according to a command from vehicle ECU 160.

When vehicle 10 takes a running-enable state, vehicle ECU 160 generates a torque command value for motor generators included in motive power output device 110 to output the generated torque command value to motive power output device 110.

Vehicle ECU 160 continually gathers, in prescribed sampling cycles, the physiological data detected by sensors 140, 142 and 144 while the user is aboard the vehicle to output the gathered physiological data to storage device 150 together with a user ID. Whether or not the user is aboard the vehicle can be determined by, for example, a seat sensor. The user ID is an identification code for identifying an occupant. Each occupant may set a user ID when getting aboard the vehicle, or may be identified by a publicly-known technique such as fingerprint recognition, vein recognition, cornea recognition, face recognition and voice recognition to be given a corresponding user ID.

Further, during charging of the power storage device in motive power output device 110 from charging station 30, vehicle ECU 160 outputs an operation command to motive power output device 110 such that motive power output device 110 carries out voltage conversion of the system power received through electric power lines ACL1 and ACL2 for charging the power storage device.

Furthermore, during charging of the power storage device, vehicle ECU 160 reads, from storage device 150, the physiological data of the occupant accumulated in storage device 150 to output the read physiological data to medical institution server 50 (FIG. 1) together with a user ID corresponding to that data, employing modem 130.

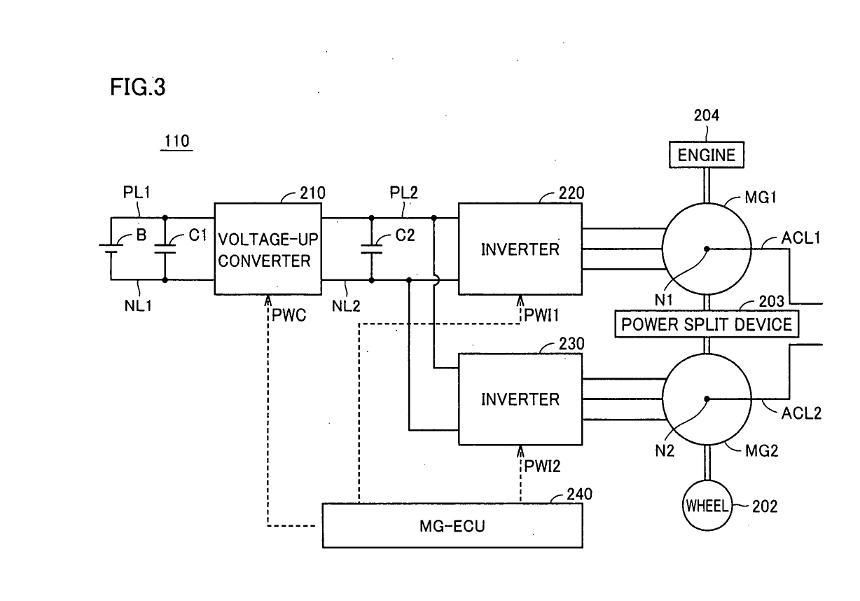

FIG. 3 is a functional block diagram of motive power output device 110 shown in FIG. 2. Referring to FIG. 3, motive power output device 110 includes an engine 204, motor generators MG1 and MG2, a power split device 203 and a wheel 202. Motive power output device 110 further includes a power storage device B, a voltage-up converter 210, inverters 220 and 230, an MG-ECU 240, capacitors C1 and C2, positive electrode lines PL1 and PL2, and negative electrode lines NL1 and NL2.

Power split device 203 is coupled to engine 204, and motor generators MG1 and MG2 to distribute motive power among them. For instance, as power split device 203, a planetary gear having three rotation shafts of a sun gear, a planetary carrier and a ring gear can be used. These three rotation shafts are connected to the rotation shaft of engine 204, motor generator MG1, and motor generator MG2, respectively.

Motor generator MG1 is incorporated in motive power output device 110 to operate as a generator driven by engine 204 and as a motor that can start engine 204. Motor generator MG2 is incorporated in motive power output device 110 as a motor for driving wheel 202 which is a driven wheel.

Each of motor generators MG1 and MG2 includes a Y-connected three-phase coil not shown, as a stator coil. Electric power line ACL1 is connected to a neutral point N1 of the three-phase coil of motor generator MG1. Electric power line ACL2 is connected to a neutral point N2 of the three-phase coil of motor generator MG2.

Power storage device B is a rechargeable DC power source, and composed of, for example, a nickel-metal hydride or lithium ion secondary battery. Power storage device B outputs DC power to voltage-up converter 210. Power storage device B is charged by receiving electric power output from voltage-up converter 210. A high-capacity capacitor may be used as power storage device B.

Capacitor C1 smoothes voltage variations between positive-electrode line PL1 and negative-electrode line NL1. Voltage-up converter 210 boosts a DC voltage received from power storage device B according to a signal PWC from MG-ECU 240 to output the boosted voltage to positive-electrode line PL2. Voltage-up converter 210 down-converts DC voltages received from inverters 220 and 230 through positive-electrode line PL2, to the voltage level of power storage device B according to signal PWC for charging power storage device B. Voltage-up converter 210 is comprised of, for example, a chopper circuit of the voltage-up/down type, or the like.

Capacitor C2 smoothes voltage variations between positive-electrode line PL2 and negative-electrode line NL2. Inverter 220 converts a DC voltage received through positive-electrode line PL2 into a three-phase AC voltage according to a signal PWI1 from MG-ECU 240 to output the converted three-phase AC voltage to motor generator MG1. Inverter 220 converts a three-phase AC voltage generated by motor generator MG1 receiving power of engine 204 into a DC voltage according to signal PWI1 to output the converted DC voltage to positive-electrode line PL2.

Inverter 230 converts a DC voltage received through positive-electrode line PL2 into a three-phase AC voltage according to a signal PWI2 from MG-ECU 240 to output the converted three-phase AC voltage to motor generator MG2. Accordingly, motor generator MG2 is driven so as to produce an indicated torque. During regenerative braking of the vehicle, inverter 230 converts a three-phase AC voltage generated by motor generator MG2 receiving the rotational force from wheel 202 into a DC voltage according to signal PWI2 to output the converted DC voltage to positive-electrode line PL2.

When power storage device B from charging station 30 (FIG. 1) is charged, inverters 220 and 230 convert the system power (single-phase AC power) supplied to neutral points N1 and N2 through electric power lines ACL1 and ACL2, respectively, into DC power according to signals PWI1 and PWI2 to output the converted DC power to positive-electrode line PL2.

Each of motor generators MG1 and MG2 is a three-phase AC motor, and comprised of, for example, a three-phase AC synchronous motor. Motor generator MG1 produces a three-phase AC voltage by means of the power of engine 204 to output the produced three-phase AC voltage to inverter 220. Motor generator MG1 produces the driving force by the three-phase AC voltage received from inverter 220 to start engine 204. Motor generator MG2 produces the driving torque for the vehicle by the three-phase AC voltage received from inverter 230. Motor generator MG2 produces a three-phase AC voltage during regenerative braking of the vehicle for output to inverter 230.

According to torque command values TR1 and TR2 from vehicle ECU 160 (FIG. 2), MG-ECU 240 generates a signal PWC for driving voltage-up converter 210, and signals PWI1 and PWI2 for driving inverters 220 and 230, respectively, to output the generated signals PWC, PWI1 and PWI2 to voltage-up converter 210, inverter 220 and inverter 230, respectively.

During charging of power storage device B from charging station 30 (FIG. 1), MG-ECU 240 generates signals PWI1, PWI2 and PWC for controlling inverter 220, inverter 230 and voltage-up converter 210, respectively, such that the system power (single-phase AC power) supplied to neutral points N1 and N2 through electric power lines ACL1 and ACL2 is converted into DC power for charging power storage device B.

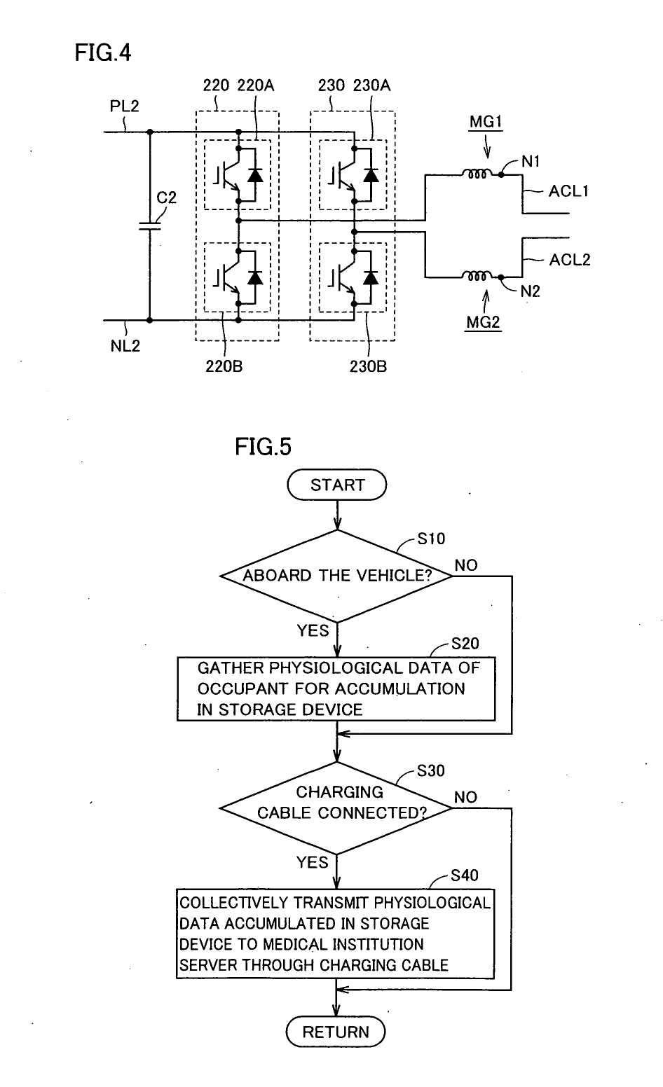

FIG. 4 illustrates a zero-phase equivalent circuit of inverters 220 and 230, and motor generators MG1 and MG2 shown in FIG. 3. In each of inverters 220 and 230 as three-phase inverters, there are eight patterns of on/off combination of six transistors. In two of the eight switching patterns, the phase-to-phase voltages become zero, and such a voltage state is referred to as a zero-voltage vector. For the zero-voltage vector, three upper-arm transistors can be regarded as being in the same switching state to each other (all on or off), and three lower-arm transistors can also be regarded as being in the same switching state to each other. Therefore, in FIG. 4, three upper-arm transistors of inverter 220 are generically shown as an upper arm 220A, and three lower-arm transistors of inverter 220 are generically shown as a lower arm 220B. Similarly, three upper-arm transistors of inverter 230 are generically shown as an upper arm 230A, and three lower-arm transistors of inverter 230 are generically shown as a lower arm 230B.

As shown in FIG. 4, this zero-phase equivalent circuit can be regarded as a single-phase PWM converter receiving system power (single-phase AC power) supplied to neutral points N1 and N2 through electric power lines ACL1 and ACL2. Then, the zero-voltage vector is changed in each of inverters 220 and 230 to control switching such that inverters 220 and 230 operate as each-phase arms of the single-phase PWM converter. Accordingly, the system power received through electric power lines ACL1 and ACL2 can be converted into DC power for output to positive-electrode line PL2.

FIG. 5 is a flow chart to describe a control structure in vehicle ECU 160 shown in FIG. 2 during charging of the power storage device. The process in this flow chart is invoked from a main routine and executed at a certain interval or each time predetermined conditions are met.

Referring to FIG. 5, vehicle ECU 160 determines whether or not a user of the vehicle is aboard the vehicle (step S10). For example, vehicle ECU 160 can determine whether or not the user is aboard the vehicle by means of a seat sensor. When vehicle ECU 160 determines that the user is not aboard the vehicle (NO in step S10), the process proceeds to step S30 without executing step S20.

When it is determined in step S10 that the user is aboard the vehicle (YES in step S10), vehicle ECU 160 continually gathers, in prescribed sampling cycles, physiological data of the occupant detected by sensors 140, 142 and 144 to output the gathered physiological data to storage device 150 together with a user ID (step S20).

Next, vehicle ECU 160 determines whether or not charging cable 20 is connected to vehicle 10 and charging station 30 (step S30). For instance, vehicle ECU 160 can determine whether or not charging cable 20 is connected to vehicle 10 and charging station 30 according to a voltage value across electric power lines ACL1 and ACL2 detected by a voltage sensor not shown.

When it is determined that charging cable 20 is connected to vehicle 10 and charging station 30 (YES in step S30), vehicle ECU 160 collectively transmits the physiological data of the occupant accumulated in storage device 150 to medical institution server 50 (FIG. 1) through charging cable 20, employing modem 130 (step S40). When it is determined in step S30 that charging cable 20 is not connected (NO in step S30), vehicle ECU 160 terminates the series of process steps without executing step S40.

Referring again to FIG. 1, the overall operation of this physiological information gathering system 1 is now described. Vehicle 10 continually gathers the physiological data of the occupant by the sensors for accumulation in the storage device. After arriving home, vehicle 10 can be connected to charging station 30 through charging cable 20 to charge the power storage device by the system power supplied from charging station 30 through charging cable 20.

During charging of the power storage device when vehicle 10 is electrically connected to charging station 30 through charging cable 20, vehicle 10 collectively transmits the physiological data of the occupant accumulated in the storage device to medical institution server 50 through charging cable 20, charging station 30, house 32 and power transmission line 40 in this order.

Specifically, sequential transmission of the physiological data continually gathered during travel to medical institution server 50 during travel employing, for example, a radio device will result in increased communication costs, whereas the present physiological information gathering system 1 is intended to collectively transmit a large amount of physiological data gathered during travel to medical institution server 50, employing charging cable 20 as a communication medium, during charging of vehicle 10.

As described above, in the present embodiment, physiological data of the occupant is continually detected by sensors 140, 142 and 144 while the occupant is aboard the vehicle, restrained on his/her seat for a long time, and the detected physiological data is stored in storage device 150. Since vehicle 10 herein allows power storage device B to be charged from charging station 30, the large amount of physiological data accumulated in storage device 150 is transmitted to an element external to the vehicle through electric power lines ACL1, ACL2 and charging cable 20 during charging of power storage device B.

Accordingly, the present embodiment enables gathering and accumulation of a large amount of physiological data of the occupant, as well as transmission of the accumulated large amount of physiological data to an element external to the vehicle. As a consequence, the large amount of physiological data gathered in the vehicle can be transmitted to medical institution server 50 to be utilized for diagnosis.

It has been described in the above embodiment that power storage device B is charged by providing the system power supplied from charging station 30 to neutral points N1 and N2 through electric power lines ACL1 and ACL2 to cause inverters 220 and 230, and motor generators MG1 and MG2 to operate as a single-phase PWM converter. However, a dedicated converter for charging power storage device B from charging station 30 may be provided separately.

While it has been described in the above embodiment that a so-called series/parallel-type hybrid vehicle in which the power of engine 4 is distributed to motor generator MG1 and wheel 2 by means of power split device 3, the present invention is also applicable to a so-called series-type hybrid vehicle in which the power of engine 4 is applied only to power generation by motor generator MG1 and the driving force for the vehicle is produced only by means of motor generator MG2. Further, the application range of the invention is not limited to a hybrid vehicle. The invention may also be applied to an electric vehicle, or a fuel cell vehicle having a rechargeable power storage device mounted thereon.

Furthermore, it has been described above that the physiological data transmitted from vehicle 10 to house 32 is transmitted to medical institution server 50 through power transmission line 40. However, data transmission from house 32 to medical institution server 50 may be achieved through another communication medium such as the Internet.

In the foregoing, electric power lines ACL1 and ACL2 correspond to “the electric power input part” according to the present invention, and motor generators MG1 and MG2, inverters 220 and 230, and voltage-up converter 210 constitute “the voltage conversion device” according to the present invention. Sensors 140, 142 and 144 correspond to “the detection device” according to the present invention, and modem 130 corresponds to “the communication device” according to the present invention. Vehicle ECU 160 corresponds to “the control unit” according to the present invention. Charging cable 20 and charging station 30 constitute “the power feeding device” according to the present invention. Medical institution server 50 corresponds to “the server” according to the present invention.

It should be construed that embodiments disclosed herein are by way of illustration in all respects, not by way of limitation. It is intended that the scope of the present invention is defined by claims, not by the description above, and includes all modifications and variations equivalent in meaning and scope to the claims.

1. A vehicle comprising:
a power storage device which is rechargeable;
an electric power input part to input electric power for charging said power storage device from a source external to the vehicle;
a voltage conversion device configured to allow the electric power input through said electric power input part to be converted into a voltage level of said power storage device for output to said power storage device;
a detection device detecting physiological data of an occupant of the vehicle;
a storage device accumulating the physiological data detected by said detection device for storage;
a communication device configured to allow communication with an element external to the vehicle through said electric power input part; and
a control unit transmitting the physiological data accumulated in said storage device to the element external to the vehicle through said electric power input part, employing said communication device.

2. The vehicle according to claim 1, wherein
said detection device continually detects said physiological data while the occupant is seated on a seat, and
when said power storage device is charged by the electric power input through said electric power input part, said control unit collectively transmits the physiological data accumulated in said storage device to the element external to the vehicle through said electric power input part, employing said communication device.

3. A physiological information gathering system comprising:
the vehicle as recited in claim 1;
a power feeding device configured to allow supply of electric power to said vehicle from a source external to said vehicle; and
a server receiving physiological data transmitted out from said electric power input part of the vehicle through said power feeding device during charging of the power storage device of said vehicle from said power feeding device.

4. The physiological information gathering system according to claim 3, wherein said power feeding device includes an electric power line electrically connected to said electric power input part during charging of said power storage device.

5. A physiological information gathering system comprising:
the vehicle as recited in claim 2;
a power feeding device configured to allow supply of electric power to said, vehicle from a source external to said vehicle; and
a server receiving physiological data transmitted out from said electric power input part of the vehicle through said power feeding device during charging of the power storage device of said vehicle from said power feeding device.
